(19) United States
(12) Patent Application Publication
(10) Pub. No.: US 2025/0000400 A1
(43) Pub. Date: Jan. 2, 2025

(54) OXYGEN SATURATION SENSOR

(71) Applicant: Drägerwerk AG & Co. KGaA, Lübeck (DE)

(72) Inventors: Thomas GRAßL, Lübeck (DE); Martha HOOT, Lübeck (DE)

(21) Appl. No.: 18/755,858

(22) Filed: Jun. 27, 2024

(30) Foreign Application Priority Data
Jun. 29, 2023 (DE) 10 2023 117 130.5

(51) Int. Cl. A61B 5/1455; A61B 5/00

(52) U.S. Cl. CPC A61B 5/14552; A61B 5/683; A61B 2560/0462; A61B 2562/12; A61B 2562/164; A61B 2562/166; A61B 2562/185

(57) ABSTRACT

An oxygen saturation sensor includes a textile basic structure extending in a direction of a longitudinal axis. The basic structure has a resiliently variable cross-sectional shape, so that the oxygen saturation sensor can be fixed to a measurement site. The oxygen saturation sensor also includes a light-emitting diode and a radiation detector. The light-emitting diode and the radiation detector are accommodated in or on the basic structure and are thus arranged in a cross-sectional plane of the basic structure.

### CROSS REFERENCE TO RELATED APPLICATIONS

This application claims the benefit of priority under 35 U.S.C. § 119 of German Application 10 2023 117 130.5, filed Jun. 29, 2023, the entire contents of which are incorporated herein by reference.

### TECHNICAL FIELD

The present invention pertains to an oxygen saturation sensor, in particular a pulse oximetric oxygen saturation sensor.

### BACKGROUND

Oxygen saturation sensors are used to determine the saturation of the blood with oxygen (oxygen saturation). A special configuration of an oxygen saturation sensor is a pulse oximetric oxygen saturation sensor.

Oxygen saturation sensors are used in intensive care and anesthesia, for example, where it is common for oxygen saturation to be measured continuously over a long period of time.

An oxygen saturation sensor has at least one light-emitting diode (LED), for example a red LED and/or an infrared LED, as well as at least one radiation detector, for example a photodiode. To determine the oxygen saturation, the absorption properties or transmission properties of the measurement site (measuring point/measuring location), for example a user's finger, can be measured and evaluated in relation to the light emitted by the at least one LED.

Depending on the desired application, oxygen saturation sensors can be reusable or configured for single use.

A reusable sensor known from DE 3 703 458 A1 has a carrier body made of silicone, rubber or polyurethane that can be deformed by material expansion, which can be placed over a patient's finger, arm or leg and adheres there by clamping forces. The sensor known from DE 3 703 458 A1 is difficult to clean and disinfect due to the complex design of the carrier body.

A sensor known from DE 69 117 861 T2 has a reusable sensor part which comprises a photodiode and which can be positioned on a patient's finger. The sensor further comprises a disposable flexible element made of non-woven fabric, which can be fixed to the finger by an adhesive layer and comprises a photoemitter.

An oximetry sensor known from U.S. Pat. No. 6,073,038 A has a foam envelope element with a mounting element (fastener), a backsheet mounting element, an LED assembly and a photodiode connected to a cable.

An oxygen saturation measuring device known from CN 2 09 122 243 U comprises a pressure sleeve. A support opening is formed in a first end of the pressure sleeve, and an oxygen saturation sensor is disposed on an inner wall of the pressure sleeve and is located near a second end of the pressure sleeve. The pressure sleeve is configured to be placed on a finger of a person to be detected. The pressure sleeve is made of elastic fabric.

US 2020/0 015 746 A1 discloses a wearable device for monitoring one or more vital signs of a human body. The device comprises a carrier adapted to be worn around an abdominal portion of a body, and an electrode assembly (electrode array) comprising a plurality of conductive electrodes, wherein the electrodes are arranged on the carrier to be brought into contact with a skin of the body in use, and wherein the electrodes are arranged to receive electrical physiological signals from the body to enable monitoring of a membrane potential of one or more muscles in the body.

US 2020/0 297 279 A1 discloses a cuff that can be worn on a user's limb. The cuff can be used to detect the shape, position and movement of the limb as well as biometric characteristics of the user.

A disadvantage of the sensors known from DE 3 703 458 A1 and DE 69 117 861 T2 is that the alignment of the photodiode and photoemitter to each other depends on the size of the finger accommodated in the sensor. This can lead to a disadvantageous signal-to-noise ratio. In addition, to avoid long-term effects such as pressure marks on a user's finger, the position of the sensor must be changed regularly, which in the case of the sensor according to DE 69 117 861 T2 makes it necessary to loosen and completely replace the flexible element. This causes the operating costs of such a sensor to increase disadvantageously.

### SUMMARY

It is an object of the invention to provide an oxygen saturation sensor which does not have these disadvantages, at least in part, and in particular to provide an oxygen saturation sensor whose measurement properties and user comfort are improved.

These and other problems are solved by an oxygen saturation sensor according to the invention.

This disclosure, including the description and the figures, provides advantageous embodiments and further details of the invention.

According to the invention, an oxygen saturation sensor is provided. The oxygen saturation sensor comprises a textile basic structure extending in the direction of a longitudinal axis, wherein the basic structure has a resiliently variable cross-sectional shape, so that the oxygen saturation sensor can be fixed to a measurement site. The oxygen saturation sensor also comprises a light-emitting diode and a radiation detector. The light-emitting diode and the radiation detector are accommodated in or on the basic structure and are thus arranged in a cross-sectional plane of the basic structure.

In the context of the invention, an oxygen saturation sensor is understood to be a sensor for determining the saturation of the blood with oxygen (oxygen saturation). Preferably, the oxygen saturation sensor is configured as a pulse oximetric oxygen saturation sensor.

A basic structure is understood to be a structure which is suitable for accommodating a light-emitting diode and a radiation detector and which can be fixed to a measurement site. A measurement site is understood to be a part of a human body that is suitable for measuring oxygen saturation, for example a finger, an arm or a leg.

According to the invention, the basic structure is configured as a textile basic structure, which is understood to mean that the basic structure is configured as a planar or spatial textile structure, i.e. comprises one or more planar or spatial textile structures or consists of such. In principle, the basic structure can have any shape, but it is preferred that the basic structure is rotationally symmetrical in relation to the longitudinal axis.

A resiliently variable cross-sectional shape is understood to mean that the basic structure has a cross-sectional shape suitable for accommodating the measurement site, wherein the basic structure or its cross-sectional shape can be changed, in particular enlarged, from its original shape by the application of force and wherein the basic structure exerts a restoring force in the deformed state in order to change the cross-sectional shape from the deformed state towards the original shape, in particular to restore it towards the original shape. In other words, the textile basic structure can be elastically deformed. As a result, the basic structure creates a fixing clamping effect on or at the measurement site if the measurement site has larger dimensions than the cross-section in its original shape. This means that the oxygen saturation sensor can be reversibly fixed to the measurement site due to the resiliently variable cross-sectional shape.

In the event that the basic structure has a non-uniform cross-sectional shape along the longitudinal axis, the cross-sectional shape refers to the cross-sectional shape that is present in the cross-sectional plane in which the LED and the radiation detector are arranged.

The basic structure can define a hollow body extending along the longitudinal axis, which has the resiliently variable cross-sectional shape at least in the cross-sectional plane. The hollow body can, for example, be cylindrical, frustoconical or a hyperboloid, i.e. bounded by a cylinder, a frustum or a hyperboloid as a boundary surface.

The oxygen saturation sensor may have one or more light-emitting diodes (hereinafter also referred to as LEDs or in plural as LEDs). Preferably, the oxygen saturation sensor has a red LED and/or an infrared LED. A red LED is understood to be an LED which is set up to emit light with a wavelength in the range from 610 nm to 760 nm, preferably with a wavelength of 660 nm. An infrared LED is understood to be an LED which is set up to emit light with a wavelength of more than 760 nm, preferably in a range from 800 nm to 1000 nm, particularly preferably with a wavelength of 950 nm.

If the oxygen saturation sensor has several LEDs, these can be configured as part of a common component or as structurally separate components.

A radiation detector is a component that is set up to measure electromagnetic radiation, namely light. Such a radiation detector is also referred to as a photodetector. The radiation detector can, for example, be configured as a photo resistor, photodiode, phototransistor, CCD sensor and/or CMOS sensor. Preferably, the radiation detector is configured as a photodiode.

The LED and/or the radiation detector can have one or more elements for influencing the beam path in the direction to or from the LED and/or in the direction to or from the radiation detector, such as one or more lenses and/or one or more reflectors.

Preferably, the longitudinal axis, the LED (or each of the LEDs) and the radiation detector (or each of the radiation detectors) enclose an angle (or an angle in each case) which is between 90 and 270 degrees, particularly preferably between 90 degrees and 180 degrees.

Preferably, the oxygen saturation sensor according to the invention is configured as a disposable oxygen saturation sensor. Alternatively, it is preferred that the oxygen saturation sensor is configured as a reusable oxygen saturation sensor, wherein it can be easily reprocessed, i.e. cleaned and disinfected, due to the textile basic structure. For example, cleaning in a washing machine is possible.

Preferably, the cross-sectional shape can be resiliently changed, in particular resiliently enlarged, by compressing the basic structure in the direction of the longitudinal axis. In this way, the handling of the oxygen saturation sensor can be improved, as the diameter of the oxygen saturation sensor can be enlarged by compressing the oxygen saturation sensor to place the oxygen saturation sensor on the measurement site and the oxygen saturation sensor can thus be placed on the measurement site without tensile or compressive stress on the measurement site. By subsequently relieving the basic structure, the cross-sectional shape is reduced again due to the restoring effect and thus creates the fixing clamping effect on the measurement site.

In a particularly preferred embodiment, the cross-sectional shape is resiliently variable by compressing the basic structure in the direction of the longitudinal axis, so that the cross-sectional shape (at least in the area of the LED and the radiation detector, i.e. at least in the cross-sectional plane) is essentially circular both in the compressed and non-compressed state.

Alternatively, preferably, the cross-section is resiliently variable by deformation of the basic structure transverse to the longitudinal axis, in particular in the cross-sectional plane. In this way, the handling of the oxygen saturation sensor can be improved, as the oxygen saturation sensor can be pushed onto the measurement site, for example onto a finger, and this pushing on causes a deformation of the basic structure transverse to the longitudinal axis, which leads to the resetting of the cross-sectional shape and consequently to the fixing clamping effect. An additional manual deformation of the basic structure to place the basic structure on the measurement site can therefore be advantageously omitted.

According to the invention, the basic structure comprises a plurality of bend-elastic yarns (flexible yarns), at least some of the yarns being selected from the group comprising plastic yarn, glass yarn and carbon yarn.

A yarn is understood to be a linear textile structure comprising one or more fibers. Preferably, the bend-elastic yarn is configured as a monofilament yarn. A plastic yarn therefore refers to a yarn that comprises one or more plastic fibers or consists of one or more plastic fibers. A glass yarn refers to a yarn that comprises one or more glass fibers or consists of one or more glass fibers. A carbon yarn refers to a yarn that comprises one or more carbon fibers or consists of one or more carbon fibers.

In this way, the elastic deformability of the basic structure can be advantageously increased, thus improving the fixation of the oxygen saturation sensor on the measurement site.

It is particularly preferable for the basic structure to consist of a plurality of bend-elastic yarns. In this way, the elastic deformability of the basic structure can be further increased.

The basic structure preferably comprises a plurality of bend-elastic yarns and a plurality of bend-flexible yarns.

The combination of bend-elastic yarns and bend-flexible yarns can provide a basic structure with high elastic deformability that is also more comfortable to wear, as bend-elastic/bend-flexible yarns (flexible yarns), i.e. soft yarns are generally more comfortable on the patient's skin. Furthermore, the properties of the basic structure can be better adapted to the conditions of use of the oxygen saturation sensor.

Preferably, the material of the plastic yarn is selected from the group comprising polypropylene, polyurethane and polyethylene.

Plastics from this group have advantageous mechanical properties at low cost.

According to the invention, the basic structure is formed as a braided, wound (coiled), woven or knitted tube.

A tube is understood to be a basic structure that is elongated along the longitudinal axis, which provides a hollow body as described above inside to accommodate the measurement site and essentially has the shape of a cylinder, a (possibly double) truncated cone or a hyperboloid.

A braided, wound, woven and/or knitted tube is understood to mean that the basic structure is obtained using a braiding process, winding process, weaving process or knitting process. The tube can be obtained both by providing one or more flat textile structures and subsequently joining edges of the one structure or joining edges of the structures to form a tube and by providing a spatial textile structure (possibly joined from partial elements). The aforementioned manufacturing processes can also be combined to obtain the tube. For example, a knitted tube can be provided with inlay threads in such a way that the inlay threads produce a braided structure. Furthermore, for example, a braided structure may have an entrained yarn.

Braided, wound, woven and/or knitted tubes offer the advantage that they are easy to manufacture and have a high deformability along the longitudinal axis and/or transverse to the longitudinal axis.

Preferably, a basic structure can be provided with a cross-sectional shape that is circular both in the compressed and non-compressed state. In particular, a centric stretching of the cross-sectional shape occurs when the shape of the basic structure is changed by compression. In other words, the angular relationship between the LED and the radiation detector is not affected by manipulation of the basic structure, e.g. by compression. This means that the orientation between the LED and the radiation detector remains constant even if the shape of the basic structure changes, for example by admission of a small finger or a large finger. Consequently, it can be ensured in this way that the measurement properties of the oxygen saturation sensor are essentially constant and therefore improved for each measurement site. An example of such a tube is also known as an extension sleeve or Chinese finger trap.

The basic structure is preferably configured as a knitted tube in such a way that the cross-section can be reset transversely to the longitudinal axis by deforming the basic structure.

The tube is preferably single-walled or multi-walled, for example double-walled.

The single-wall configuration offers the advantage of easy manufacturability. The multi-walled design offers the advantage of increased restoring forces.

The multi-walled design can be achieved, for example, by inverting the tube over itself several times. Optionally, the butt edges formed by the inversion can be joined, e.g. by welding.

Preferably, the basic structure is open on one or both sides.

If the basic structure is open on one side (open only on one side), the shielding of the inside of the oxygen saturation sensor against stray light can be improved compared to the basic structure that is open on both sides.

If the basic structure is open on both sides, the cleanability of the oxygen saturation sensor can be improved compared to the basic structure open on one side, provided that the oxygen saturation sensor is not configured as a disposable oxygen saturation sensor.

Preferably, the oxygen saturation sensor may further comprise an opaque structure surrounding at least a portion of the basic structure and/or at least a portion of the plurality of bend-elastic yarns to reduce incidence of light from the environment of the oxygen saturation sensor towards the radiation detector.

An opaque structure is a structure that is configured to reduce the transmission of light through the basic structure.

In this way, the signal-to-noise behavior of the oxygen saturation sensor and thus its measurement properties can be improved.

In a preferred embodiment, the opaque structure can be configured as a second layer that at least partially, preferably completely, surrounds the basic structure on its outer side.

Such a structure can, for example, be integral with the basic structure, for example in the form of a coating on the basic structure, or be configured as a separate layer, for example as a cover on the basic structure, in particular as a knitted textile cover.

Another option for providing an opaque structure is to make the basic structure partly from bend-elastic yarn and partly from light-reducing yarn. At least one part of the plurality of bend-elastic yarns is thus surrounded on at least one side by light-reducing yarns as an opaque structure. For example, a number of bend-elastic yarns can be inserted into a structure knitted from light-reducing yarn, or the basic structure can be woven from bend-elastic yarn and light-reducing yarn, for example by forming some or all of the weft threads or warp threads with the light-reducing yarn.

The basic structure is particularly preferably configured as a two-part knitted tube, which has a first inner tube and a second outer tube. The first inner tube can comprise a plurality of yarns that ensure good wearing comfort and the second outer tube can comprise a plurality of yarns (opaque bend-elastic yarns) that provide an opaque structure.

Preferably, the LED and/or the radiation detector is mounted in or on the basic structure by welding, in particular spot welding, gluing, inserting or sewing.

In this way, the LED and/or the radiation detector can be connected to the basic structure without adversely affecting the deformability of the basic structure.

Preferably, the LED and/or the radiation detector are accommodated as part of one or more flexible printed circuit boards in or on the basic structure.

In this way, the LED and/or the radiation detector can be connected to the basic structure without adversely affecting the deformability of the basic structure.

The LED and/or the radiation detector are particularly preferably incorporated as part of one or more flexible printed circuit boards in or on the basic structure by welding, in particular spot welding, gluing or sewing. In a further variant, the printed circuit board is woven or woven into the basic structure as part of the latter.

Preferably, the oxygen saturation sensor also has a fixing means (fixative/fastener) which is set up to fix the oxygen saturation sensor on or at the measurement site.

This can be used to further increase the fixing force of the oxygen saturation sensor on the measurement site.

The fixing means can, for example, be configured as a band with a Velcro fastener or as a rubber ring.

The oxygen saturation sensor can also have other elements, for example for data processing and communication. For example, the oxygen sensor can have a control unit such as a circuit or a microprocessor for controlling the LED and the radiation detector. The control unit can, for example, be configured to receive and process measurement signals from the radiation detector and/or store them in a memory unit. Furthermore, for example, the oxygen saturation sensor can have circuits for controlling and/or reading out the LED and/or the radiation detector. The circuits may include, for example, one or more amplifiers, resistors, capacitors, filters, A/D converters and the like. The circuits can be configured as part of the control unit or as separate components. Furthermore, for example, the oxygen saturation sensor can have a data interface, such as a wired or wireless interface, for sending and/or receiving data. For example, the interface can be used to transmit the measurement signals from the radiation detector and/or measurement data stored in a memory unit to an external receiver. Furthermore, for example, the oxygen saturation sensor can have an energy source such as an accumulator or a battery to provide electrical energy for operating the oxygen saturation sensor. Additionally or alternatively, the oxygen saturation sensor may have a power interface (wired or wireless) for receiving electrical power for operating the oxygen saturation sensor. All of the aforementioned elements can be configured as part of a common circuit board, for example as part of the flexible circuit board or circuit boards described above.

Preferably, the oxygen saturation sensor has a color coding that indicates its intended use. For example, a reusable oxygen saturation sensor can be configured in a first color and a non-reusable oxygen saturation sensor can be configured in a second color that is different from the first color.

These and other features and advantages can also be seen from the following description of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1a is a longitudinal sectional view of an embodiment of an oxygen saturation sensor according to the invention;

FIG. 1b is a cross-sectional view through the cross-sectional plane of an embodiment of an oxygen saturation sensor according to the invention;

FIG. 2a is a front view of an embodiment of an oxygen saturation sensor according to the invention before deformation;

FIG. 2b is a front view of an embodiment of an oxygen saturation sensor according to the invention after deformation;

FIG. 3a is a front view of a further embodiment of an oxygen saturation sensor according to the invention before deformation;

FIG. 3b is a front view of an oxygen saturation sensor according to the invention after deformation;

FIG. 4 is a detailed top view of an embodiment of a woven tube according to the invention;

FIG. 5a is a detailed longitudinal sectional view of an embodiment of a woven tube according to the invention;

FIG. 5b is a detailed longitudinal sectional view of an embodiment of a woven tube according to the invention with an opaque structure;

FIG. 5c is a detailed longitudinal sectional view of a further embodiment of a woven tube according to the invention with opaque structure;

FIG. 6 is a front view of a further embodiment of an oxygen saturation sensor according to the invention with fixing means; and

FIG. 7 is a longitudinal sectional view of another embodiment of an oxygen saturation sensor according to the invention.

### DESCRIPTION OF PREFERRED EMBODIMENTS

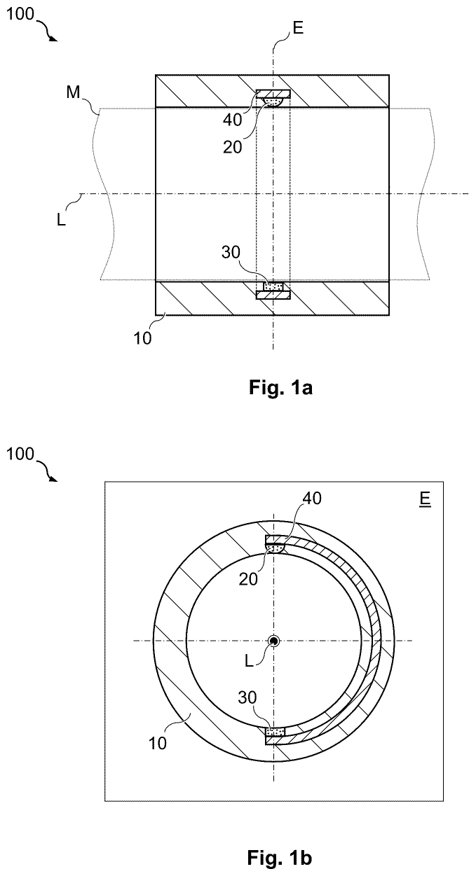

Referring to the drawings, FIGS. 1a to 3b and 6-7 show examples of oxygen saturation sensors 100 according to the invention.

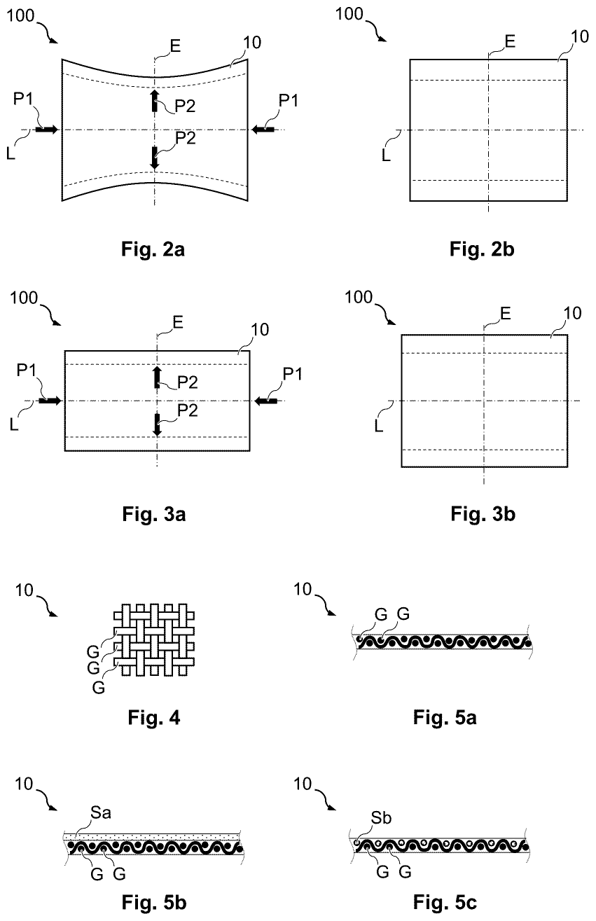

Each oxygen saturation sensor 100 according to the invention comprises a textile basic structure 10 extending in the direction of a longitudinal axis L. The basic structure 10 according to the invention can, for example, be cylindrical in shape, as shown in the embodiment examples according to FIGS. 1a, 1b, 3a, 3b, 6 and 7. In another embodiment, the basic structure 10 is configured as a hyperboloid, as shown in FIGS. 2a, 2b. Other forms of the basic structure 10 are also possible.

The basic structure 10 of each embodiment has a resiliently variable cross-sectional shape, so that the oxygen saturation sensor 100 can be fixed to a measurement site M. The measurement site M, which can be a user's finger, for example, is indicated schematically in FIG. 1a.

It is preferable that the cross-sectional shape of the basic structure 10 can be reset by compressing the basic structure 10 in the direction of the longitudinal axis L. This is indicated in the two embodiment examples according to FIG. 2a and FIG. 3a, which each show an oxygen saturation sensor 100 in its basic state, i.e. before deformation. If the basic structure 10 is compressed along the longitudinal axis L, i.e. deformed, as indicated by the pair of arrows P1-P1, the cross-sectional shape changes, in particular the cross-sectional shape becomes larger (see FIGS. 2b and 3b in the deformed state). In this state, the oxygen saturation sensor 100 can be placed on the measurement site M particularly gently. By relieving the load on the resiliently variable basic structure 10, it is fixed to the measurement site M by clamping forces.

Alternatively, in each embodiment example, it is preferred that the cross-sectional shape can be changed by deformation of the basic structure 10 transversely to the longitudinal axis L. This is indicated as an alternative in the two embodiment examples according to FIG. 2a and FIG. 3a by the pairs of arrows P2-P2. If the basic structure 10 is deformed transversely to the longitudinal axis L, as indicated by the pair of arrows P2-P2, the cross-sectional shape changes, in particular the cross-sectional shape becomes larger (see FIGS. 2b and 3b). The restoring forces caused by the deformation fix the basic structure 10 to the measurement site M by clamping forces.

Each oxygen saturation sensor 100 according to the invention further comprises a light-emitting diode 20 (LED) and a radiation detector 30, as shown in FIGS. 1a and 1b and not shown in the other figures for the sake of clarity. Each oxygen saturation sensor 100 according to the invention may comprise more than one LED 20 and/or more than one radiation detector 30.

The LED 20 and the radiation detector 30 are accommodated in or on the basic structure 10 and are thus arranged in a cross-sectional plane E of the basic structure 10, as can be seen in FIGS. 1a and 1b.

The LED 20 and the radiation detector 30 can, as shown in the example according to FIGS. 1a, 1b, be arranged with their outer surface flush with a boundary surface of the hollow body provided by the basic body 10 or, in an embodiment not shown, project beyond the boundary surface of the hollow body into the latter (partially or completely), i.e. not be flush with the boundary surface.

The way in which the LED 20 and the radiation detector 30 are accommodated in or on the basic structure 10 can be configured as desired. However, it is preferred in all embodiments that the LED 20 and/or the radiation detector 30 are accommodated (indirectly or directly) in or on the basic structure 10 by welding, gluing or sewing. In a further variant, it is preferred that LED 20 and/or radiation detector 30 are inserted into pockets of the basic structure 10 configured for this purpose.

It is further preferred in all embodiments that the LED 20 and/or the radiation detector 30 are accommodated as part of one or more flexible printed circuit boards 40 in or on the basic structure 10, as shown in FIGS. 1a, 1b. In the example shown, the printed circuit board 40 is embedded in the basic structure 10, but it is also possible to arrange the printed circuit board 40 flush with the boundary surface of the hollow body or to arrange the printed circuit board 40 on an outer side of the base body 10. The printed circuit board 40 can be accommodated in or on the basic structure 10 by welding, gluing, inserting or sewing in order to accommodate the LED 20 and/or the radiation detector 30 indirectly by welding, gluing, inserting or sewing in or on the basic structure 10.

It is in accordance with the invention that the basic structure 10 is formed as a braided, wound, woven and/or knitted tube. An example of a woven tube is shown in FIG. 4 as a broken-out detailed view in plan view of the basic structure 10. The woven tube is formed by a plurality of yarns G, which can be of the same or different types.

Although the fabric according to FIG. 4 is shown with warp yarns and weft yarns running at right angles to each other, angular relationships between warp yarns and weft yarns or between the yarns forming the tube are possible which deviate from this. In the context of the invention, it was recognized in this respect that the deformability of the basic structure 10 is advantageously higher the more the angle included between the two yarn directions deviates from 90°. Particularly in the case of braided tubes, angles that deviate significantly from 90° can be achieved.

The woven tube according to FIG. 4 is shown as a broken-off detailed view in longitudinal section in FIG. 5a.

According to the invention, the basic structure 10 comprises a plurality of bend-elastic yarns (flexible yarns) G, at least some of the yarns G being selected from the group comprising plastic yarn, glass yarn and carbon yarn. Preferably, the material of the plastic yarn is selected from the group comprising polypropylene, polyurethane and polyethylene.

The tube formed by the basic structure 10 can be single-walled, as shown in FIGS. 1a to 3b. FIG. 7 shows that the tube formed by the basic structure 10 can be multi-walled.

Furthermore, it is possible in all embodiments, and shown in FIGS. 1a to 3a, that the basic structure 10 can be open on both sides. Not shown, but possible in all embodiment examples, is that the basic structure 10 is open on one side. In this case, one of the end faces of the basic structure 10 can be closed by a separate element or by a corresponding closed formation of the textile basic structure 10 during the manufacturing process.

In all embodiments, it is preferred that the oxygen saturation sensor 100 comprises an opaque structure Sa, Sb surrounding at least a portion of the basic structure 10 and/or at least a portion of the plurality of bend-elastic yarns G to reduce light incidence from the environment of the oxygen sensor 100 towards the radiation detector 30.

An embodiment example of such an opaque structure Sa is shown in FIG. 5b. In this case, the opaque structure Sa is configured as a second layer Sa that at least partially, preferably completely, surrounds the basic structure 10. Such a layer Sa can, for example, be provided as an integral part of the basic structure 10, such as a coating, or as a separate layer, for example as a cover.

Another embodiment of an opaque structure Sb is shown in FIG. 5c. In this case the basic structure 10 comprises both bend-elastic yarns G and light-reducing yarns (opaque yarns) Sb. In the example shown, the tube is a fabric that comprises both light-reducing yarns Sb and bend-elastic yarns G.

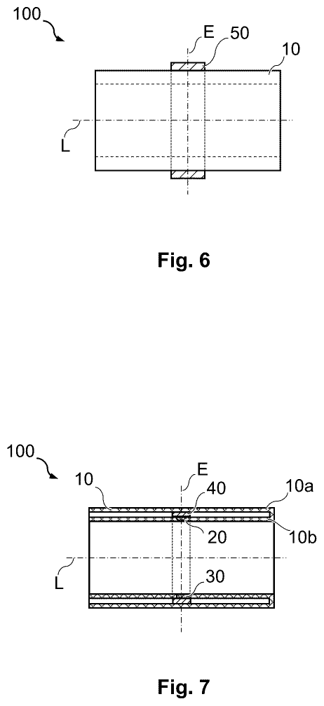

In some embodiments, it is preferred that the oxygen saturation sensor 100 additionally has a fixing means (fixative/fastener) 50, which is set up to fix the oxygen saturation sensor 100 on or at the measurement site M. An example of such an oxygen saturation sensor 100 is shown in FIG. 6. The fixing means 50 can be integrally connected to the oxygen saturation sensor 100, for example sewn to it. Alternatively, the fixing means 50 may be provided separately from the oxygen saturation sensor 100 and may be connected to it for fixing the oxygen saturation sensor 100 to the measurement site M. The fixing means 50 can be, for example, a rubber ring or a band provided with a Velcro fastener, which can be looped around the oxygen saturation sensor 100.

It is preferred and shown in FIG. 7 that the tube formed by the basic structure 10 can be multi-walled, for example by inverting the tube 10 over itself. This results in a basic structure 10 with several walls 10a, 10b. As shown in FIG. 7, such a multi-walled basic structure 10 can be configured to accommodate the printed circuit board 40 in between.

While specific embodiments of the invention have been shown and described in detail to illustrate the application of the principles of the invention, it will be understood that the invention may be embodied otherwise without departing from such principles.

### LIST OF REFERENCE SYMBOLS

10 Basic structure
10a First wall
10b Second wall
20 Light emitting diode (LED)
30 Radiation detector
40 Printed circuit board
50 Fixing means (fixative)
100 Oxygen saturation sensor
E Cross-section level
G, Gs Yarn
L Longitudinal axis
M Measurement site
P1-P1 Compression direction pair of arrows
P2-P2 Deformation direction pair of arrows
Sa, Sb Opaque structure

What is claimed is:

1. An oxygen saturation sensor comprising:
a textile basic structure extending in a direction of a longitudinal axis, the basic structure comprising a resiliently variable cross-sectional shape configured such that the oxygen saturation sensor can be fixed to a measurement site,
a light-emitting diode; and
a radiation detector,
wherein the light-emitting diode and the radiation detector are accommodated in or on the basic structure and are thus arranged in a cross-sectional plane of the basic structure,
wherein the basic structure is formed as a braided, wound, woven and/or knitted tube, and
wherein the basic structure comprises a plurality of flexible yarns, at least a portion of the yarns being selected from the group comprising plastic yarn, glass yarn and carbon yarn.

2. An oxygen saturation sensor according to claim 1,
wherein the cross-sectional shape is resiliently variable by compressing the basic structure in the direction of the longitudinal axis, or
wherein the cross-sectional shape is resiliently variable by deforming the basic structure transversely to the longitudinal axis.

3. An oxygen saturation sensor according to claim 2, wherein the material of the plastic yarn is selected from the group comprising polypropylene, polyurethane and polyethylene.

4. An oxygen saturation sensor according to claim 2, wherein the tube is single-walled or multi-walled.

5. An oxygen saturation sensor according to claim 2, wherein the basic structure is open on one side or is open on both sides.

6. An oxygen saturation sensor according to claim 2, further comprising:
an opaque structure surrounding at least a part of the basic structure and configured to reduce light incidence from the environment of the oxygen sensor towards the radiation detector and/or
opaque flexible yarns wherein at least a part of the plurality of flexible yarns comprise the opaque flexible yarns to reduce light incidence from the environment of the oxygen sensor towards the radiation detector.

7. An oxygen saturation sensor according to claim 2, wherein the light-emitting diode and/or the radiation detector are accommodated in or on the basic structure by welding, gluing, inserting or sewing.

8. An oxygen saturation sensor according to claim 2, further comprising a printed circuit board arrangement comprising one or more flexible printed circuit boards, wherein the light-emitting diode and/or the radiation detector are accommodated as part of the printed circuit board arrangement.

9. An oxygen saturation sensor according to claim 2, further comprising a fixing means which is arranged to fix the oxygen saturation sensor on or at the measurement site.

10. An oxygen saturation sensor according to claim 1, wherein the material of the plastic yarn is selected from the group comprising polypropylene, polyurethane and polyethylene.

11. An oxygen saturation sensor according to claim 1, wherein the tube is single-walled or multi-walled.

12. An oxygen saturation sensor according to claim 1, wherein the basic structure is open on one side or is open on both sides.

13. An oxygen saturation sensor according to claim 1, further comprising:
an opaque structure surrounding at least a part of the basic structure and configured to reduce light incidence from the environment of the oxygen sensor towards the radiation detector and/or
opaque flexible yarns, wherein at least a part of the plurality of flexible yarns comprise the opaque flexible yarns to reduce light incidence from the environment of the oxygen sensor towards the radiation detector.

14. An oxygen saturation sensor according to claim 13, further comprising a printed circuit board arrangement comprising one or more flexible printed circuit boards, wherein the light-emitting diode and/or the radiation detector are accommodated as part of the printed circuit board arrangement.

15. An oxygen saturation sensor according to claim 13, wherein the light-emitting diode and/or the radiation detector are accommodated in or on the basic structure by welding, gluing, inserting or sewing.

16. An oxygen saturation sensor according to claim 1, wherein the light-emitting diode and/or the radiation detector are accommodated in or on the basic structure by welding, gluing, inserting or sewing.

17. An oxygen saturation sensor according to claim 1, further comprising a printed circuit board arrangement comprising one or more flexible printed circuit boards, wherein the light-emitting diode and/or the radiation detector are accommodated as part of the printed circuit board arrangement.

18. An oxygen saturation sensor according to claim 1, further comprising a fixing means which is arranged to fix the oxygen saturation sensor on or at the measurement site.

19. An oxygen saturation sensor comprising:
a textile basic structure comprising a plurality of flexible yarns that are braided, wound, woven and/or knitted forming a tube extending in a direction of a longitudinal axis, at least a portion of the yarns being selected from the group comprising plastic yarn, glass yarn and carbon yarn, the basic structure comprising a resiliently variable cross-sectional shape configured to fix the oxygen saturation sensor to a measurement site with the cross-sectional shape being resiliently variable by compressing the basic structure in the direction of the longitudinal axis and/or being resiliently variable by deforming the basic structure transversely to the longitudinal axis;
a light-emitting diode; and
a radiation detector, the light-emitting diode and the radiation detector being accommodated in or on the basic structure so as to be arranged in a cross-sectional plane of the basic structure.

20. An oxygen saturation sensor according to claim 19, further comprising:
an opaque structure surrounding at least a part of the basic structure and configured to reduce light incidence from the environment of the oxygen sensor towards the radiation detector and/or
opaque flexible yarns, wherein at least a part of the plurality of flexible yarns comprise the opaque flexible yarns to reduce light incidence from the environment of the oxygen sensor towards the radiation detector.
